(19) United States
(12) Patent Application Publication
(10) Pub. No.: US 2010/0010530 A1
(43) Pub. Date: Jan. 14, 2010

(54) Balloon Dilation for Implantable Prosthesis

(75) Inventor: Eugene Y. Rhee, San Diego, CA (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 12/373,667

(22) PCT Filed: Jul. 16, 2007
(86) PCT No.: PCT/US07/16090

§ 371 Date: Jan. 13, 2009

Related U.S. Application Data
(60) Provisional application No. 60/891,009, filed on Feb. 21, 2007.

(51) Int. Cl. A61M 29/00

(52) U.S. Cl. 606/192; 604/96.01

(57) ABSTRACT

Balloon dilation addresses the dangers of blunt instrument dilation or surgical dissection by placing a balloon within soft tissue and inflating the balloon to displace the soft tissue in a manner to create a cavity or space for receipt of an implantable device. A prosthesis balloon dilation catheter set facilitates a surgical procedure to implant semi-rigid and/or inflatable prostheses.

### PRIORITY

This application claims priority to PCT Application No. PCT/US2007/016090, filed Jul. 16, 2007, which claims priority to U.S. Provisional Application Ser. No. 60/831,009, filed Jul. 14, 2006, the entire contents of which are incorporated herein by reference.

### BACKGROUND

The field relates to instruments and methods for forming free space cavities in soft tissue in which devices are to be implanted. More specifically, the field includes use of balloon inflation to form a cavity, a pocket, or a tunnel in soft tissue for implantation of a device or an element of an apparatus. For example, the field includes an instrument with an inflatable balloon for forming an elongate cavity in a corpus cavernosum of a penis for implantation of a prosthetic inflatable cylinder to aid in treating erectile dysfunction

The body of the penis consists of a pair of corpora cavernosa dorsally, and a single corpus spongiosum ventrally. These structures are surrounded by fascia and skin. The three structures are composed of erectile tissue enclosed in a dense fibroelastic connective tissue—the tunica albuginea. The erectile tissue consists of a fine network of fibroelastic tissue, the spaces of which are lined with endothelium. When these spaces are filled with blood, an erection ensues. When viewed in cross section, the corpora cavernosa are seen to be divided by a connective tissue septum. The septum is complete in the proximal part of the penis but incomplete in the distal portion. The corpora cavernosa are two spongy paired cylinders contained in the thick envelope of the tunica albuginea. Their proximal ends, the crura, originate at the undersurface of the puboischial rami as two separate structures but merge under the pubic arch and remain attached to the glans (the tip of the penis). It is within these two corporal bodies that the penile implant cylinders are surgically inserted to mimic the rigid erection necessary for coitus. Posteriorly, the corpora cavernosa diverge and end as the crura of the penis which are located in the superficial space of the perineum and are attached to the inferior pubic rami. Anteriorly, the corpora terminate as rounded ends; they play no part in formation of the glans of the penis. The posterior part of the corpus spongiousum, which contains the urethra, forms the bulb of the penis, which is also located in the superficial part of the perineum. Anteriorly, the corpus spongiosum ends as the expanded glans penis. The urethra enters the deep surface of the bulb after piercing the perineal membrane and goes through the entire length of the corpus spongiosum, and in the glans of the penis lies nearer the ventral than the dorsal surface.

The penile cavernosal smooth muscle and the smooth muscle of the arteries of the penis are primarily responsible for the quality of erection. The smooth muscles are contracted in the flaccid state allowing minimal arterial flow. Sexual stimulation triggers the release of neurotransmitters from the cavernous nerves resulting in relaxation of the contracted smooth muscles, followed by: 1) dilation of the arterioles and arteries by increased blood flow; 2) trapping of the incoming blood by the expanding sinusoids; 3) compression of the subtunical venular plexuses between the tunica albuginea and the peripheral sinusoids, reducing the venous outflow; 4) stretching of the tunica to its capacity, which encloses the emissary veins between the inner circular and outer longitudinal layers and further decreases the venous outflow to a minimum; 5) an increase in the intracavernous pressure (maintained at 100 mm Hg), which raises the penis from the dependent position to the erect state (the full erection phase); and 6) a further pressure increase (to several hundred mm of mercury) with contraction of the ischiocavernosus muscles (rigid erection phase). Erection thus involves sinusoidal relaxation, arterial dilation, and venous compression.

Prosthetic implants to restore erectile function were introduced in the 1930's. Early prosthetic implants utilized rib cartilage or acrylic beneath Buck's fascia of the penis. Infection, erosion, pain, and poor function explained poor success rates in the past. The introduction of intracorporal cylinders of semirigid and inflatable types significantly improved patient satisfaction rates in the 1970's. The current penile implants have proven to provide durable patient satisfaction rates, excellent cosmetic results, function and reliability. The penile implants have successfully addressed penile reconstruction, Peyronie's disease, and priapism.

The penile prosthesis has been in existence over the past 3 decades with two types of penile prosthetics developed: the semi-rigid and the inflatable. The terms “semi-rigid” and “malleable” are often used interchangeably in the urology literature. A semi-rigid implant may have a stainless steel member with a woven wire core surrounded by a polymer type material such as silicone. The cylinders formed thereby are typically 13 mm in diameter and are supplied in different lengths as well as diameters with rear tip polymer cap extenders for accurate sizing. A typical mechanical semi-rigid prosthetic consists of articulating segments of high molecular weight polyethylene threaded over a wire attached on either end by a spring mechanism. The first inflatable penile prosthesis, made from Dacron-reinforced silicone elastomer, consisted of four parts: an inflation pump, a deflation pump, paired non-distensible cylinders, and a rectangular fluid reservoir. In the initial report, it was described in use in five patients. Modifications of the inflatable penile prosthesis since the first reported inflatable penile prosthesis in 1974 ultimately led to a variety of inflatable penile prosthetics. They include one, two, or three pieces, are filled with normal saline, and are constituted of two distensible penile cylinders, a pump and a reservoir.

It is estimated that the prevalence of erectile dysfunction is 52% in non-institutionalized men aged 40 to 70 years of age. Such conditions can range in severity from partial to complete inability to achieve a rigid erection sufficient enough to coitus. It is well known that the prevalence of erectile dysfunction increases significantly with age.

The known causes of erectile dysfunction are psychogenic, neurogenic, vasculogenic, and drug-induced. The aging process alone is a major cause of erectile dysfunction.

Treatment of erectile dysfunction widely varies from conservative measures to aggressive surgical implantation of penile prosthesis. Lifestyle changes such as cessation of smoking and exercise have been known to improve the quality of erections. Change of recognized medications affecting erectile function is another treatment option. Psychosexulal therapy for psychogenic causes is another treatment option. Oral medications that act centrally and peripherally are now available as treatment options. The more popular oral medications include the phosphodiesterase inhibitors (Viagra®, Levitra®, Cialis®). More invasive options include direct injection of vasoactive drugs into the cavernosal bodies. Finally, in particular special cases where vascular injury is quite evident from trauma, direct vascular repair is available. Today, the most popular surgical therapy for erectile restoration is implantation of implantable penile prosthesis (also called “penile implants”).

Penile implant products are available, for example, from American Medical Systems, Minnetonka, Minn. and Coloplast, Minneapolis, Minn. Both companies manufacture semi-rigid and inflatable prostheses. The inflatable devices are either self-contained cylinders or multi-piece devices. Selection of the appropriate device for the individual patient is largely based on three considerations: the patient's preference, the cost of the device, and the surgeon's preference. Representative products include semi-rigid prostheses sold under the Dura™ brand and inflatable prostheses sold under the AMS700™ brand by American Medical Systems. Representative products also include semi-rigid prostheses sold under the Acu-Form® brand and inflatable prostheses sold under the Titan® brand by Coloplast. See also the emedicine article entitled “Penile Prosthesis Implantation” by R. A. Santucci, et al., updated Mar. 8, 2006 at the emedicine website.

With respect to the surgical implantation of penile prosthesis, the procedures are considered to be highly technically challenging and necessitate the surgeon to possess a considerable body of experience in the techniques. It is well known in the art of surgical implantation of a penile prosthesis that considerable risk attends the initial incision, exposure of the tunica albuginea surface of the corpus cavernosum, dilation of the corporal space, estimation of diameter and length of each of the two corporal bodies, placement of the penile prosthesis cylinders within the corpora and closure of the coporotomies.

The primary objective in implantation of these prostheses is to provide the patient with a phallus capable of coitus. The prostheses involve placing a rod (semi-rigid or inflatable) into each corpora cavernosa. The rods maintain some degree of rigidity within the corpora if a semi-rigid prosthesis is used, or inflate with sterile fluid within the device to distend the chambers in the case of inflatable prosthesis.

The implantation procedure typically involves a two to three hour surgery under general anesthesia. A 4 cm penoscrotal incision is made in the midline at the penoscrotal junction and the corpora cavernosa is dissected free bilaterally. A longitudinal 2 cm incision is made in the tunica albuginea about 1 cm lateral to the urethra. The corpora are then serially dilated both proximally and distally by using a succession of incrementally thicker dilators. To avoid urethral injury, dilation must occur by “hugging” the lateral walls of the corpora to avoid iatrogenic injury to the urethra or septum (cross-over).

Serial dilation involves the use of a succession or series of elongate, blunt, rigid, metal rods (“dilators”). First a dilator with a small diameter is pushed through the incision to make a corresponding elongate cavity or tunnel in the corpora that extends distally from the incision almost to the glans. The first dilator is then withdrawn and a second one of a somewhat larger diameter is pushed through the cavity in order to widen it, and so on until the cavity is wide enough to receive one implant cylinder. This maneuver typically requires eight progressively larger dilators for each corpus. Two parallel cavities or tunnels are formed, one in each of the corpora.

Typically, serial dilation for penile prosthesis occurs with a #7 French dilator initially, followed by dilation with a succession of dilators, up to, typically, a #14 French dilator. Careful dilation is critical in this procedure. Insufficient dilation distally may lead to inadequate support of the glans by the prosthesis. Aggressive uncontrolled dilation can lead to septal perforation in the midline of the phallus or urethral injury. Proximal dilation that is too vigorous can lead to proximal perforation of the corpora or migration of the prosthesis. Next, measurements are made of the proximal and distal corporal lengths. Rear tip extenders are used to add length to the cylinders as needed proximally. The cylinders are inserted into the corpora and the corporotomy is closed with suture. Once the corporotomies are closed, the cylinders are inflated or the rods are inspected to assess the quality of the erection and ensure the cylinders are bilaterally even within the glans. If the inflatable penile prosthesis is used, the next step is implantation of the reservoir in the paraveiscal space. This is accomplished by palpating the external ring and bluntly perforating the transversalis fascia. The reservoir is placed within this paravesical space. Another maneuver is to make a lower right quadrant incision (known in the urology literature as a “counter incision”) and place the reservoir in the preperitoneal pocket underneath the rectus muscle. The scrotal pump of the inflatable penile prosthesis is implanted within a subdartos pouch within the penoscrotal incision. The skin incisions are subsequently closed.

Another approach is to make an infrapubic incision to expose bilateral corpora and make the respective corporotomies. The cylinders are thus implanted in a similar fashion as the penoscrotal approach.

It is clear that the most difficult step in penile prostheses implantation is the actual dilation of the corpora which, for safety and a successful outcome, requires accurate measurements of the corpora as dilation proceeds in order to avoid urethral injury, proximal and distal perforation, septal perforation of the corpora spongiosum, and other possible harm. Additionally, the number of sequential dilations (typically eight dilations) increases the chances for injury or perforation. Gentle preservation of remnant corpora spongiosum is not accomplished with blunt serial dilation. The surgical technique of dilating laterally attempts to help prevent urethral injury. The Dilamezinsert tool was patented 20 years ago to facilitate dilation bluntly. However, this instrument can cause septal perforation and is not used routinely.

Other urologic prostheses require creation of differently shaped cavities conforming to anatomical features that are difficult to access. For example, implantation of an artificial sphincter requires creation of arcuate space around the circumference of the bulbous urethra in order to position a cuff around the urethra. The use of surgical instruments in circumferentially in dissecting the soft tissue of the bulbous urethra to create such space always poses a risk of damage to the urethra, especially at the dorsal side or 12:00 position.

Dilation of soft tissue to create a cavity where none existed is a fraught, but necessary step for implantation of multiple surgical prostheses for a variety of indications. There is a substantial need for the surgical implantation of such prosthesis to be made easier and safer in respect of dilation and creation of soft tissue space. These objectives are accomplished with balloon dilation of the soft tissue to create space therein for receipt and retention of an implantable element.

In some cases, there is a need to quickly assess the adequacy of the space created by inflation of the balloon, measure its dimensions, and assess implant placement therein. Provision of measuring indicia on the balloon carrier meets these needs.

### SUMMARY

A balloon dilation instrument addresses the dangers of serial dilation or surgical dissection by placing a balloon within soft tissue and inflating the balloon to displace the soft tissue in a manner to create a cavity or space for receipt of an implantable device. In some aspects, the instrument may be marked in order to accurately and quickly make measurements for placement of the implantable device.

In one embodiment, balloon dilation addresses the dangers of serially dilating the corpora cavernosa by placing an elongate balloon laterally within the corpora and inflating the balloon to gently displace the spongiosal tissue medially. This quickly dilates the corpora and forms the cavity. Additionally, the balloon has marked centimeter measurements so when the balloon is fully inflated, the measurements are accurately and quickly made.

In another embodiment, balloon dilation addresses the dangers of injury to the urethra by placing an elongate balloon circumferentially around the back of the bulbous urethra and inflating the balloon to displace the periutheral tissue circumferentially. This dilates the periutheral tissue and forms the cavity.

I contemplate that, once the skilled medical practitioner apprehends how an inflated balloon can dilate soft tissue to create a cavity therein, he or she will then appreciate how dilation inflation can replace lengthy, tedious, and risky procedures by which such cavities have heretofore been created. For example, it is generally known that corporal serial dilation is a technically challenging procedure that is not commonly encountered in everyday urologic practice. This is also known to be the most common technique leading to many intra-operative complications. However, balloon technology and the surgical implantation of balloon devices are very common in surgical practice, and are used by urologists in many aspects of percutaneous stone management and to clear strictures in the ureter and the urethra. Thus, once they learn the techniques and tools taught in this application, urologists should exhibit a high degree of comfort and confidence when using this technology to dilate soft tissue, such as penile tissue, for implantation of prostheses and other devices.

### BRIEF DESCRIPTION OF THE DRAWINGS

FIGS. 1A, 1B, and 2-6 illustrate elements of an instrument set for balloon dilation of soft tissue in the penis.

FIG. 1A illustrates a hollow point dilator; FIG. 1B is a cross-section of the dilator;

FIG. 2 illustrates a needle sheath introducer with sharp, beveled distal end and proximal bulbous blunt end;

FIG. 3 illustrates a stiff angled glide wire with a lubricous coating;

FIG. 4 illustrates a distal balloon catheter;

FIG. 5 illustrates an inflation syringe; and

FIG. 6 illustrates a proximal balloon catheter.

FIGS. 7A and 7B are illustration of soft tissue in which balloon dilation is used to create space for a prosthetic penile apparatus.

FIG. 8 illustrates the implanted prosthetic penile apparatus.

FIGS. 9 and 10 illustrate elements of an instrument set for dilation of soft tissue around the bulbous urethra or the anal canal.

FIG. 9 illustrates a blunt curved needle.

FIG. 10 illustrates a balloon instrument.

FIG. 11 illustrates tissue surrounding a bulbous urethra in which balloon dilation is used to create space for a prosthetic urethral sphincter apparatus.

FIG. 12 illustrates the implanted prosthetic sphincter apparatus.

FIG. 13 illustrates an implanted prosthetic sphincter apparatus in which balloon dilation is used to create space for a cuff of apparatus.

### DETAILED DESCRIPTION OF THE PREFERRED EMBODIMENTS

Balloon dilation in this specification refers to the inflation of a balloon in soft tissue to create, or to complete the creation of a cavity in the soft tissue where none formerly existed, wherein the cavity is created to receive a medical implant. Preferably, the cavity is created with a shape corresponding to the shape of an implant which is disposed and retained in the cavity. A balloon dilation set is a set of one or more instruments that enable the insertion of a balloon into soft tissue for the purpose of creating, or completing the creation of a cavity in the soft tissue where none existed before, wherein the cavity is created to receive a medical implant. The embodiments described in this specification are directed toward implantation of prosthesis devices or elements thereof, but this is not intended to, and does not exclude the application of the illustrated principles to creation of cavities in soft tissues for other medical implants such as therapeutic and measurement devices.

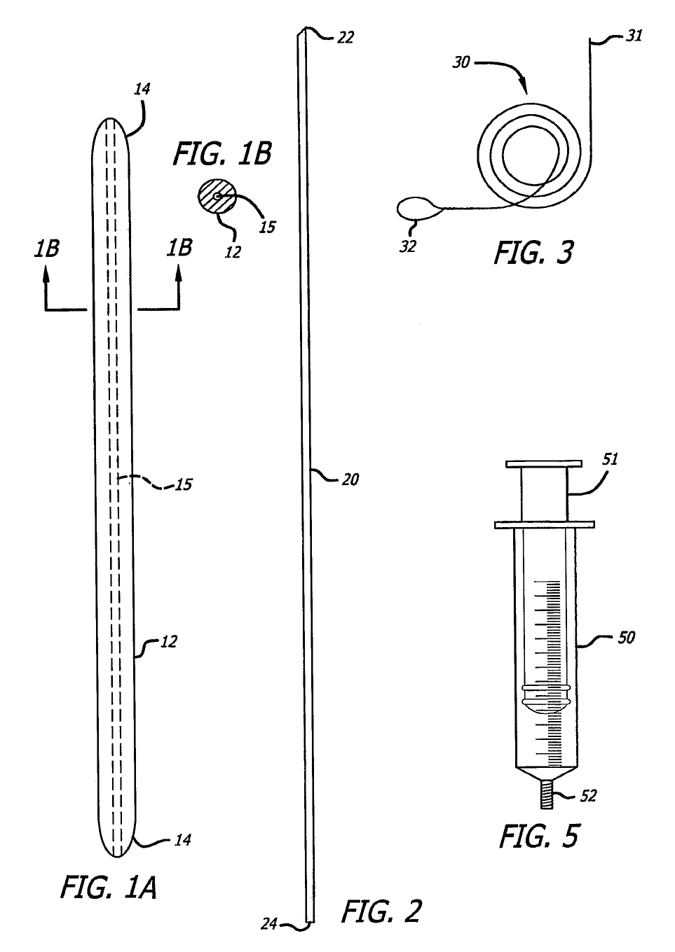

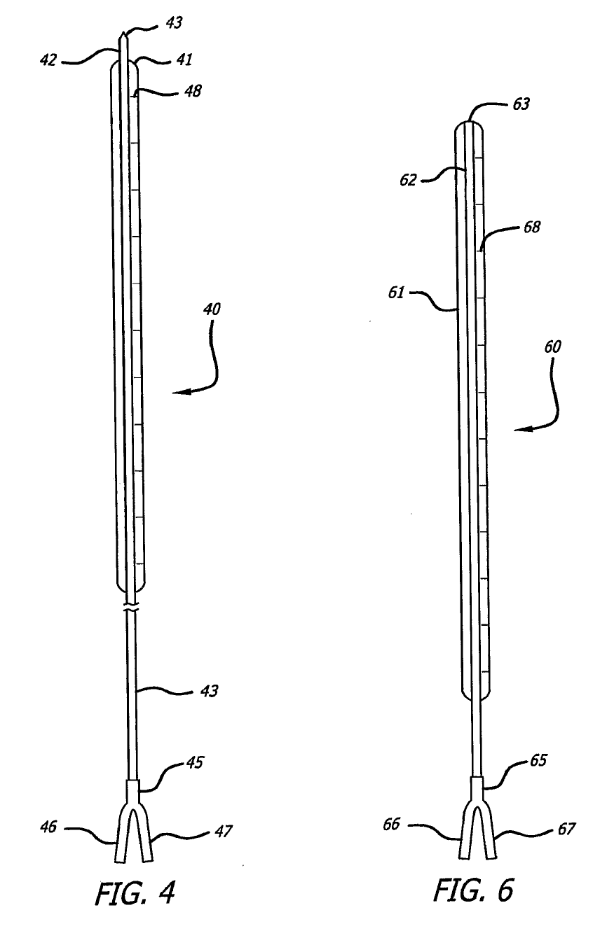

In one embodiment, balloon dilation is used in creating at least one cavity in a corpus cavernosum for implantation of a penile prosthesis, either semi-rigid or inflatable. An instrument set for balloon dilation of soft tissue (“a balloon dilation set”) to receive a penile prosthesis apparatus includes elements illustrated in FIGS. 1A, 1B, and 2-5. Preferably, although not necessarily, this balloon dilation set includes at least one dilator (FIGS. 1A and 1B), at least one sheath introducer (FIG. 2), at least one glide wire (FIG. 3), at least one distal balloon instrument (FIG. 4), and at least one inflation syringe (FIG. 5). In some aspects, this balloon dilation set further includes at least one proximal balloon catheter (FIG. 6). The set supports methods that allow for both creation of new spaces in at least one corpus cavernosum as well as dilation of scarred spaces therein to allow implantation of a penile prosthesis.

With reference to FIGS. 1A and 1B, a dilator 12 may be, for example, an elongate, rigid, hollow point, 7 French, plastic or steel dilator that is tapered on both ends 14. The dilator 12 may be straight or slightly curved like a Hager dilator. The length of the dilator 12 should be sufficient to reach through a corpus cavernosum, from a penoscrotal (or infrapubic) entry point to a point near the glans; for example, the dilator may be at least 20 cm in length. Preferably, the dilator 12 is hollow, having a central bore 15, with a small diameter. For example, the diameter of the bore 15 may be about 5 mm.

As seen in FIG. 2, a needle sheath introducer 20 may be, for example, a hollow, 20 gauge, 20 cm long, stainless steel or plastic needle sheath introducer with sharp, beveled distal end 22 and proximal bulbous blunt end 24.

As illustrated in FIG. 3, a glide wire (also called a guide wire) 30 may be a 0.888 mm (0.035 in) diameter, relatively stiff, wire of a length sufficient to reach through the introducer 20, with excess length left outside; for example, the glide wire 30 may be 75 cm in length. Preferably, the glide wire 30 has a lubricous coating, a straight distal end 31, and a proximal end 32 in which a small loop or eye is formed.

As per FIG. 4, a distal balloon dilation instrument may include an instrument with the construction of a distal balloon catheter, such as the distal balloon catheter 40 illustrated in FIG. 4, preferably, two such distal balloon instruments are provided. Preferably, the catheter 40 has a balloon 41 mounted distally on a tube 42 with a distal tip 43. Preferably, the tube 42 is a double-walled tube which includes an air supply path for inflating the balloon 41 and a bore extending to the distal tip 43. A connector 45 on the proximal end of the tube 42 includes a first port 46 in communication with the bore and a second port 47 in fluid communication with the air supply path. The balloon 41 has a length and a diameter appropriate to the size of a patient receiving a penile prosthesis. For example, the balloon may be about 20 cm long with either a 10 F or a 14 F diameter. In some aspects, the balloon 41, or, if the balloon is transparent, the surface of the tube 42 within the balloon, may have a series of uniformly-spaced marks 48 disposed longitudinally of the tube 42 as an aid to making measurements as described below. The distal balloon instrument is of sufficient stiffness that, with a lubricous coating, allows for smooth advancement within the corpora of the penis.

FIG. 5 illustrates a source of pressurized air which may be connected to a distal balloon instrument for inflating a balloon thereon. For inflation of catheter-mounted balloons such as the balloon 41 illustrated in FIG. 4, the source of pressurized air may be constituted, for example, of a Leveen inflation syringe 50 with a plunger 51 and a tip 52 which connects to the port 47 of the distal balloon catheter 40.

At least one proximal balloon instrument, such as the proximal balloon catheter 60 illustrated in FIG. 6, is provided; preferably, two such proximal balloon instruments are provided. The catheter 60 has a balloon 61 mounted proximally on a tube 62 with a distal tip 63. Preferably, the tube 62 is a double-walled tube which includes an air supply path for inflating the balloon 61 and a bore extending to the distal tip 63. A connector 65 on the proximal end of the tube 62 includes a first port 66 in communication with the bore and a second port 67 in fluid communication with the air supply path. The balloon 61 has a length and a diameter appropriate to the size of a patient receiving a penile prosthesis. For example, the balloon may be about 10 cm long with either a 10 F (for smaller anatomic corpora) or a 14 F diameter. In some aspects, the balloon 61, or, if the balloon is transparent, the surface of the tube 62 within the balloon, may have a series of uniformly-spaced marks 68 disposed longitudinally of the tube 62 as an aid to making measurements as described below

One example of a balloon dilation set for use in creating parallel cavities in corpora cavernosa includes distal balloon catheters having a lubricous coating. Each distal balloon catheter is hollow so as to slide over a 20 gauge needle sheath introducer (18 gauge). Each distal balloon catheter has a sharply tapered tip so as to slide along a needle introducer and out the glans penis. Marks spaced at, for example, 1 cm, are labeled on the distal balloon catheters. The distal balloon catheters are 20 cm in length, each with a distal port and balloon port. The proximal balloon catheters are 10 cm in length. All of the distal and proximal balloon catheters may come in 2 or more balloon sizes, for example, 10 and 14 French.

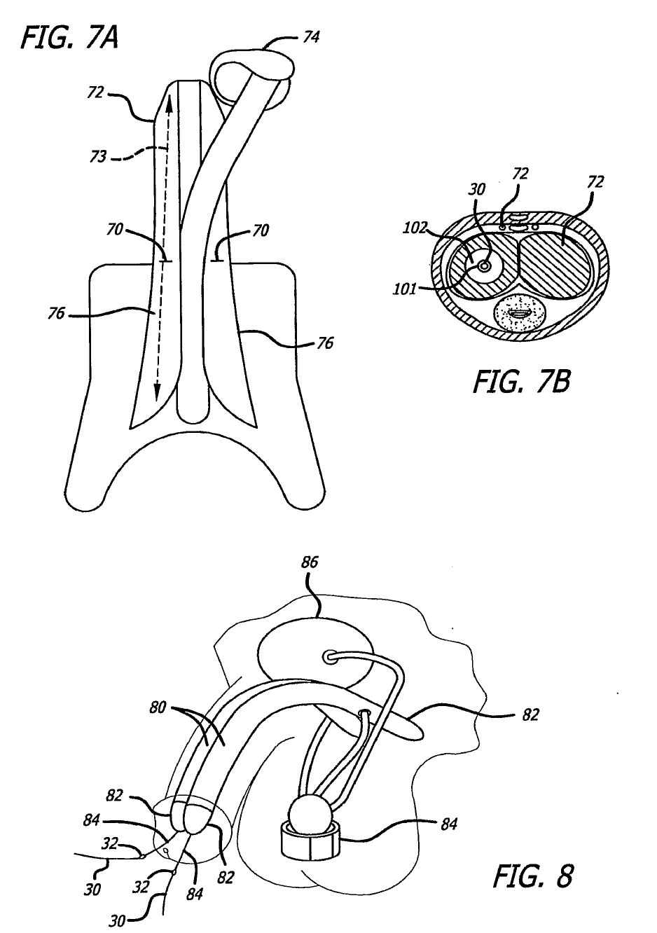

With reference to FIG. 7A, in using a penile prosthesis balloon dilation set such as one including distal and proximal balloon catheters in the example above, a surgeon makes a traditional penoscrotal or infra-pubic incision, and then makes the traditional respective corporotomies in the corpora. For example, the locations of respective coporotomies via penoscrotal incisions are indicated by 70. The surgeon may then create an initial opening in a corpus cavernosum 72 by advancing a small (7 French) hollow point dilator through an incision 70 and into the corpus distally (in the direction 73) until the end of the dilator is palpated within the glans penis 74 in the traditional fashion. A 20 gauge needle sheath introducer is then advanced through the dilator, which is held in position within the respective corpus 72, until the distal end of the introducer is pushed out through the glans 74. A 0.888 mm glide wire is threaded through the needle sheath introducer in the dilator until the distal end of the wire emerges through the distal end of the introducer and is clamped outside of the glans 74. The dilator is then pulled proximally, out through the incision 70, and removed, leaving the needle sheath introducer and the wire positioned within the corpus 72. A first distal balloon catheter is advanced, distal end first, toward the glans 74, over the proximal portion of the wire, and then over the needle introducer and the wire (the lubricious coating of the balloon catheter facilitates advancement forward) until the distal tip of the catheter tube is detected at the glans 74. The needle introducer sheath is then retracted from the corpus, leaving the distal balloon catheter with the glide wire extending through the bore thereof in the corpus.

With reference to FIGS. 7A and 7B, a Leveen inflation syringe is attached to the balloon port of the catheter and is activated. The inflation syringe has a pressure gauge to accurately confirm proper inflation. The first balloon is held secure manually as full inflation occurs. As it inflates, the balloon expands circumferentially and compresses the soft tissue of the corpus, creating generally cylindrical space therein for implantation of a prosthesis cylinder. See FIG. 7B in this regard. The syringe is locked on full inflation and the distal measurements are made by observing the marks on the balloon. If desired, this balloon may be left intact and inflated within one corpus while attention is then turned to the other corpus. The same technique is then performed for the other corpus cavernosum with an identical set of elements to place a second distal balloon catheter. The second balloon is inflated with a second inflation syringe and the second inflation syringe is locked. With both balloons inflated the effect of an inflatable penile prosthetic is mimicked and an immediate assessment of the dilated corpora can be made. In this regard, inflation of the distal balloons erects the penis in much the same manner as an inflatable penile prosthesis. The penis is inspected while erected by the first and second balloons to assess the quality of dilation in the corpora cavernosa, and to gauge the proposed positions of the penile prosthetic cylinders to be placed. Then, the first and second balloons are deflated. The two distal catheters and the two needle introducers may be retracted along the glide wires, out of the corpora cavernosa through the incisions 70, and off of the glide wires.

Next, a hollow 7 French dilator may used to traditionally dilate the proximal end 76 of each corpus. The blunt bulbous end of a hollow spinal needle may be inserted through the dilator until the bone of the inferior rami is encountered. The dilator is removed and a proximal balloon catheter with no tip extending from the tip of the balloon is advanced over the needle. The needle is removed and the proximal catheter balloon is inflated using an inflation syringe. Immediate measurements are made proximally and this is repeated for the other proximal corpus end 76. With the proximal portions of the corpora cavernosa thus dilated and evaluated, the needles and proximal catheters are removed, and the cylinders of the implants are placed in the traditional manner and the remaining part of the penile implantation is completed.

With reference to FIG. 8, a complete inflatable penile prosthesis apparatus is shown, implanted in a male patient. The prosthesis apparatus includes inflatable stiffening cylinders 80. In some aspects, once the cavities for the cylinders 80 have been formed by the exemplary method described above, the balloon catheters may be removed by sliding them proximally over the and off of the glide wires. The glide wires may be left in the distal portions of the corpora cavernosa in order to assist in placement of the stiffening cylinders. In this regard, it is known to provide an eyelet 82 in a solid portion at the end of each stiffening cylinder 80, to which a suture 84 is threaded to provide a line to grip and pull the cylinder home in the cavity created by inflation of a balloon. In this regard, each suture 84 may be tied or secured to the proximal end 32 of a respective glide wire 30, after removal of the balloon catheter therefrom. Pulling the distal end of the glide wire 30 out through the glans 74 will pull the prosthesis cylinder coupled to its proximal end into place in the cavity created in the corpus. When the cylinder is seated, the suture is cut and, with the glide wire 30, pulled out of the corpus through the glans.

In another embodiment, balloon dilation is used in creating a circumferential cavity around the urethra for implantation of the inflatable cuff of an artificial sphincter therein. An artificial urethral sphincter apparatus includes an elastic reservoir to store an inflating fluid, an inflatable cuff, and a control pump to control the flow of inflating fluid between the reservoir and the cuff. These elements are connected by tubing, and all elements are implanted in the body. The urethral cuff is implanted around the bulbous urethra to keep the urethra closed. Squeezing the pump implanted in the soft tissue of the scrotum or labium empties fluid from the cuff into the elastic reservoir, and opens the urethra for urination. Afterwards the fluid slowly refills the cuff, closing the urethra. For an understanding of the operation and implantation of an artificial urethral sphincter, see the article by D. S. Elliott and D. M. Barrett entitled “The Artificial Genitourinary Sphincter” in DIGITAL UROLOGY JOURNAL, downloaded Jul. 11, 2007. Implantation of an artificial urethral sphincter apparatus requires creation of one or more cavities in soft tissue where elements of the apparatus are implanted. One such cavity must be formed around the urethra in order to implant the cuff.

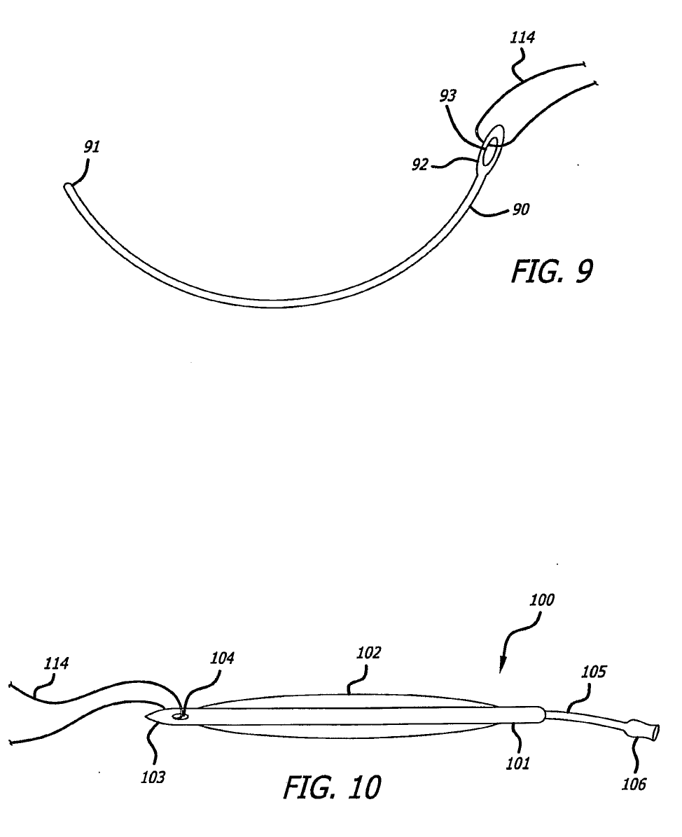

A balloon dilation set for dilation of the soft tissue around the urethra includes elements illustrated in FIGS. 9 and 10. Preferably, although not necessarily, the instrument set includes a curved needle (FIG. 9), a distal balloon catheter (FIG. 10), and an inflation syringe (FIG. 5) As seen in FIG. 9, a curved needle 90 has a distal end 91 with a blunt tip and an end 92 with an eyelet 93. In FIG. 10, a distal balloon catheter 100 includes a hollow tube 101 with a balloon 102 mounted thereon. The balloon has a length and an inflation width appropriate to the dimensions of the patient. Typically, for an adult male, the balloon may be about 10 cm long and 10 French when inflated. The distal end 103 of the tube 101 extends beyond the balloon 102, preferably for about 2 cm. The distal end includes an eyelet 104 and ends in a sharpened tip. A connector 105 with a balloon port 106 is coupled to the proximal end of the tube 101.

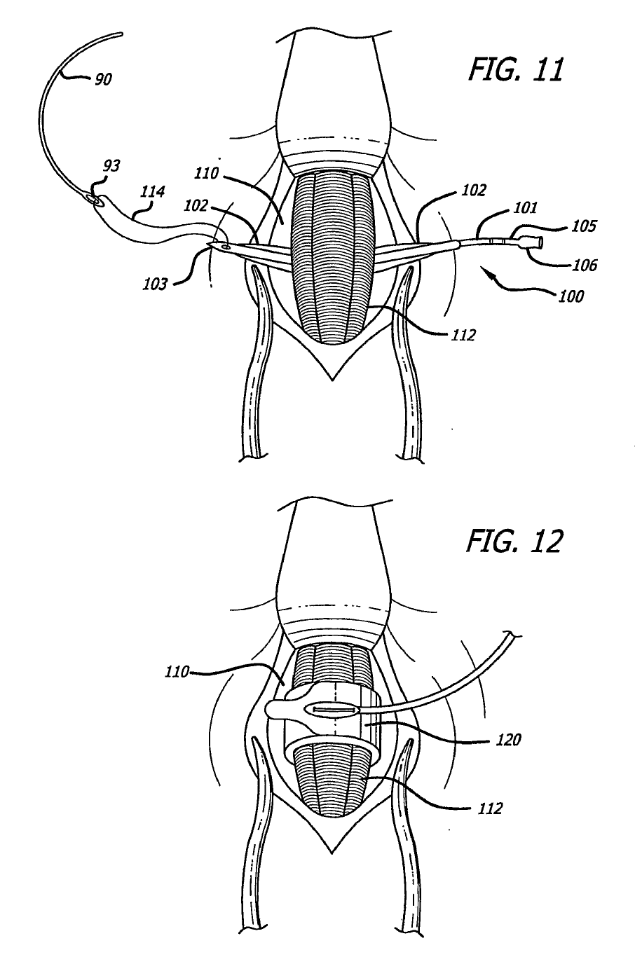

Placement of a cuff for an artificial sphincter requires opening a circumferential cavity posteriorly around the bulbous urethra. See the article by D. S. Elliott and D. M. Barrett cited previously. With reference to FIGS. 9 and 11, the bulbocavernous muscle 110 is opened along the front of the bulbous urethra 112 of a patient in the lithotomy position. In order to seat the cuff, a suture 114 is threaded through the eyelet 93 of the curved needle 90 and through the eyelet 104 in the distal end of the balloon sphincter 100. The needle 90 is oriented such that its concave side faces the bulbous urethra 112, and, in this orientation, is passed, blunt distal end 91 first through the undissected soft tissue, along one side, around the rear, and back along and out the other side of the bulbous urethra 112, pulling the suture 114 through the soft tissue. As the suture 114 is pulled, the balloon catheter 100 follows, distal end 103 first, along the path traveled by the needle 90 around the back of the bulbous urethra, until it is in the position seen in FIG. 11. At this position, the suture 114 is removed from the eyelet 105 the balloon 102 is inflated, dilating the soft tissue to create the circumferential cavity around the bulbous urethra 112. Again, with the balloon 102 inflated, the effect of an inflatable sphincter prosthetic is mimicked and an immediate assessment of the dilated corpora can be made. The balloon catheter 100 may have markings as described above to enable measurement of the circumferential cavity. The balloon 102 is then deflated, and pulled back out of the circumferential cavity by the coupler 105. The inflatable cuff 120 of the artificial sphincter is passed through the circumferential cavity thus created around the back of the bulbous urethra 112, and its ends are joined around the front of the bulbous urethra as seen in FIG. 12. Other balloon instruments may be provided to create cavities by balloon dilation in soft tissue for other elements of the artificial urethral sphincter.

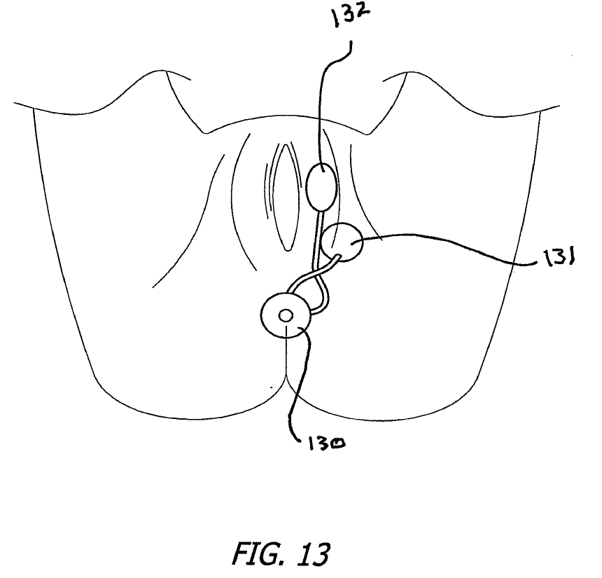

In another embodiment, balloon dilation is used in creating a circumferential cavity around the anal canal for implantation of the inflatable cuff of an artificial sphincter therein. With reference to FIG. 13, an artificial anal sphincter is a surgically implanted device constituted of an inflatable cuff 130, a pressure regulating elastic reservoir 131 and a control pump 132. The rectal cuff 130 is implanted around the anal canal to keep the canal closed. Squeezing the pump 132 implanted in the soft tissue of the labium (or scrotum) empties fluid from the cuff 130 into the elastic reservoir 131, and opens the anal canal for defecation. Afterwards the fluid slowly refills the cuff 130, closing the anal canal. Surgical implantation of the cuff is accomplished via a perineal incision under the vagina (or scrotum), isolating the rectal sphincter circumferentially in a similar fashion to that of the aforementioned urethra. The posterior aspect of the rectum is a risky dissection similar to the dorsal urethra whereas using the proposed balloon catheter safely and efficiently allows a circumferential empty space to be developed for the anal cuff.

Placement of a cuff for an artificial anal sphincter requires opening a circumferential cavity posteriorly around the anal canal. See the article by J. Christiansen and B. Sparso entitled “Treatment of Anal Incontinence by an Implantable Prosthetic Anal Sphincter” in ANNALS OF SURGERY, April 1992, pp. 383-386. In this regard, either a single incision is made along the perineum or two are made on either side of the anus of a patient in the lithotomy position, and an opening is made in the soft tissue along the anal canal. In order to seat the cuff, a circumferential opening is created posteriorly around the anus using an instrument set such as is illustrated in FIGS. 9 and 10. That is to say, a blunt needle tethered thereto by a suture to the sharp tip of a balloon catheter. Then, working through the opening, the needle is passed circumferentially under and around the rectum far enough below the bowel to avoid injury thereto, and is pulled out of the soft tissue. The movement of the blunt needle as just described causes the balloon catheter to be pulled circumferentially under and around the rectum to a position where the balloon catheter is untethered from the needle and the balloon is inflated to create the circumferential cavity posteriorly to the rectum. The balloon catheter may have markings as described above to enable measurement of the circumferential cavity. The balloon is then deflated, and pulled back out of the circumferential cavity by the coupler mounted to its proximal end. The inflatable cuff of the artificial sphincter is passed through the circumferential cavity thus created around the back of the rectum, and its ends are joined around the front of the rectum as seen in FIG. 13. Other balloon instruments may be provided to create cavities by balloon dilation in soft tissue for other elements of the artificial anal sphincter, which are shown in FIG. 13.

The novel tools and methods disclosed and illustrated herein may suitably be practiced in the absence of any element or step which is not specifically disclosed in the specification, illustrated in the drawings, and/or exemplified in the embodiments of this application. Furthermore, although the balloon dilation instruments and methods have been described with reference to presently preferred embodiments, it should be understood that various modifications can be made without departing from the spirit of the principles set forth herein. Accordingly, my invention is limited only by the following claims.

1. A dilation instrument set for creating a cavity for an implant in soft tissue, comprising:
a hollow point dilator;
a needle sheath introducer with a sharp distal end and blunt proximal end;
a glide wire;
at least one distal balloon instrument;
at least one proximal balloon instrument; and
at least one inflation syringe.

2. The set of claim 1, wherein the distal balloon instrument is a distal balloon catheter.

3. The set of claim 2, further including measurement markings on the distal balloon catheter.

4. A dilation instrument for creating a cavity in soft tissue in which a prosthesis device is to be placed, the instrument including a tube with distal and proximal ends, a balloon mounted distally on the tube, an air supply path in the tube for inflating the balloon, connector on the proximal end of the tube including a port in communication with the air supply path, and a lubricious coating on the instrument, wherein the balloon has a length and a diameter appropriate to the size of a patient receiving the prosthesis.

5. The dilation instrument of claim 4, further including measurement markings on the balloon or on the tube.

6. The dilation instrument of claim 4, wherein the tube is a double-walled tube which includes the air supply path for inflating the balloon and further includes a bore extending to the distal tip and the connector on the proximal end of the tube includes the port in communication with the air supply path and further includes a port in communication with the bore.

7. A method of dilation for placement of an inflatable prosthesis in tissue, comprising:
advancing a hollow point dilator through the tissue until palpated at a distal portion of the tissue;
advancing a hollow introducer through the dilator while the dilator is in position within the tissue so that the tip of the introducer is pushed out through the distal portion;
threading a glide wire is threaded through the introducer until an end of the glide wire protrudes through the distal portion;
clamping the end of the end of the glide wire;
removing the dilator while leaving the introducer and the glide wire in place;
advancing a first distal balloon catheter with a distal tip and a colored safety zone tab on the distal tip over the introducer and the glide wire into the tissue, distal tip first, until the colored safety zone tab emerges through the distal portion;
retracting the needle introducer sheath from the glide wire; and
inflating the first distal balloon within the tissue.

8. A method of dilation for placement of a penile prosthesis in penile tissue, comprising:
advancing an elongate balloon distally in a corpus cavernosum;
inflating the elongate balloon to open a tubular passage within the corpus cavernosum for receipt of a penile implant.

9. A dilation instrument set, comprising:
a curved needle with a blunt distal end; and
a balloon dilation instrument with a sharp distal end.

10. The dilation instrument set of claim 9, the balloon dilation instrument including a tube with a sharp distal end and a proximal end, a balloon mounted distally on the tube, an air supply path in the tube for inflating the balloon, a connector on the proximal end of the tube including a port in communication with the air supply path, and a lubricious coating on the instrument.

11. The dilation instrument set of claim 10, further including an eyelet in the sharp distal end for receiving a suture to tether the balloon dilation instrument to the curved needle.

12. The dilation instrument set of claim 11, further including measurement markings on the balloon dilation instrument.

13. A method of dilation for placement of an inflatable prosthesis in tissue, comprising:
advancing a curved needle on a circumferential path through the tissue;
advancing a balloon catheter, distal tip first, on the circumferential path into the tissue; and
creating a circumferential space in the tissue by inflating a balloon on the distal balloon catheter within the tissue.

14. A method of dilation for placement of an inflatable sphincter prosthesis in tissue around a bulbous urethra, comprising:
circumferentially disposing a balloon in the tissue around the bulbous urethra;
inflating the balloon to open a circumferential cavity around the bulbous urethra.

15. The method of claim 14, wherein circumferentially disposing includes:
advancing a curved needle on a circumferential path through the tissue; and,
pulling a balloon catheter, distal tip first, on the circumferential path into the tissue.

16. A method of dilation for placement of an inflatable sphincter prosthesis in tissue around a rectum, comprising:
circumferentially disposing a balloon in the tissue around the rectum;
inflating the balloon to open a circumferential cavity around the rectum.

17. The method of claim 16, wherein circumferentially disposing includes:
advancing a curved needle on a circumferential path through the tissue; and,
pulling a balloon catheter, distal tip first, on the circumferential path into the tissue.

18. A method of dilation for placement of an implant in soft tissue, comprising:
advancing a balloon dilation instrument with a distal tip through the soft tissue; and,
inflating a balloon on the balloon dilation instrument within the soft tissue to create a cavity in the soft tissue;
wherein, the cavity is shaped to receive an implant.

19. The method of claim 18, further including removing the balloon dilation instrument from the space and then placing the implant in the cavity.

20. The method of claim 18, wherein the balloon dilation instrument is a distal balloon catheter, further including, before advancing:
initially creating an opening in the soft tissue with a dilator; and,
placing an introducer with a coaxial glide wire in the opening;
wherein, advancing includes advancing the distal balloon catheter on the introducer, followed by removing the introducer.

21. The method of claim 20, wherein the implant is a semi-rigid or an inflatable penile prosthesis.

22. The method of claim 18, wherein the balloon instrument is a distal balloon catheter, further including, before advancing:
initially creating a curved path in the soft tissue with a curved needle;
wherein, advancing includes advancing the distal balloon catheter along the curved path, following the needle.

23. The method of claim 22, wherein the implant is an inflatable sphincter prosthesis.
